(19) United States
(12) Patent Application Publication
(10) Pub. No.: US 2006/0045851 A1
(43) Pub. Date: Mar. 2, 2006

(54) Oral care composition comprising essential oils

(75) Inventors: Jamesina Anne Fitzgerald, Trenton, OH (US); Nancy Jean Flennoy, Cincinnati, OH (US); Christina Lula Johnson, Germantown, OH (US); Kevin Ian Trevor Wright, Frimley Green (GB)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/217,274

(22) Filed: Sep. 1, 2005

Related U.S. Application Data
(60) Provisional application No. 60/606,557, filed on Sep. 2, 2004.

(51) Int. Cl. A61K 8/97; A61K 9/68; A61K 36/752; A61K 36/534

(52) U.S. Cl. 424/48; 424/58; 424/736; 424/739; 424/747

(57) ABSTRACT

An essential oil composition comprising at least two essential oil from Group A which includes mint and spice essential oils and at least one essential oil from Group B which includes citrus essential oils. This essential oil composition can effectively kill and remove oral microbials which are correlated to the formation of biofilm in the oral cavity. This essential oil composition can be combined with a variety of oral carriers and actives.

### CROSS REFERENCE TO RELATED APPLICATION

This application claims the benefit under 35 USC 119(e) to U.S. application Ser. No. 60/606,557, filed Sep. 2, 2004.

### FIELD OF THE INVENTION

The present invention relates to oral care compositions that comprise an effective amount of an essential oil composition which kills and inhibits microbials from the oral cavity and disrupts the biofilm on the hard and soft tissues in the oral cavity. Further, this invention relates to a method of treating or preventing oral conditions. Compositions of the present invention are suitable for use by humans or animals.

### BACKGROUND OF THE INVENTION

Oral malodor, plaque, gingivitis, periodontal disease and discoloration of the teeth are all undesirable conditions that affect many people. It is generally believed that the presence of microbials in the mouth contribute to these conditions. As a result, there is considerable consumer demand for oral care products that effectively kill microbials on the teeth and other surfaces of the oral cavity. Regular brushing with traditional oral care products may not be sufficient to remove all of the food and oral microbial deposits that adhere to the oral surfaces.

Recent developments in oral care products include the use of natural essential oils to provide a wide range of benefits. Conventionally, herbal based products, such as essential oils, have been included in oral care products for flavor as well as cleansing, freshening, and in some cases anti-microbial benefits. However, a need still exists for essential oils compositions that can provide improved anti-microbial benefits, and in particular essential oil compositions which destroy the microbial correlated to the formation of biofilm within the oral cavity.

### SUMMARY OF THE INVENTION

An essential oil composition comprising two or more essential oils selected from Group A including spearmint, peppermint, a botanical of the Labiatae family, carvone, cinnamon, cassia, clove, anise, ginger, pepper, nutmeg, allspice, and coriander; and one or more essential oils selected from Group B including lemon, lime, orange, grapefruit, tangerine, mandarin, Ugli fruit, bergamot, and tomato; at a ratio of Group A essential oils to Group B essential oils of from about 2:1 to about 10:1.

An oral care composition comprising two or more essential oils selected from the following Group A including: spearmint, peppermint, a botanical of the Labiatae family, carvone, cinnamon, cassia, clove, anise, ginger, pepper, nutmeg, allspice, and coriander, and one or more essential oils selected from the following Group B including: lemon, lime, orange, grapefruit, tangerine, mandarin, Ugli fruit, bergamot, and tomato; wherein the ratio of Group A essential oils to Group B essential oils is from about 2:1 to about 10:1. The oral care composition further comprises an oral care carrier selected from the group consisting of dentifrice, chewable dentifrice tablet, gels, rinses, edible films, candy, confectionary, gums, and lozenges.

An oral care composition comprising an essential oil composition, wherein said essential oil composition comprises at least one mint essential oil, at least one spice essential oil, and at least one citrus essential oil; wherein said essential oils are added at a ratio of from about 6 mint: 1 spice: 1 citrus to about 2 mint: 1 spice: 1 citrus. The oral care composition further comprises an oral care carrier selected from the group consisting of dentifrice, gels, rinses, edible films, candy, confectionary, gums, and lozenges; and an oral care active.

### DETAILED DESCRIPTION OF THE INVENTION

### A. DEFINITIONS

The term “orally active” as used herein means a material that provides either a cosmetic, prophylactic or therapeutic benefit within the oral cavity.

The term “teeth”, as used herein, is meant to include natural teeth, dentures, dental plates, fillings, caps, crowns, bridges, dental implants, and the like, and any other hard surfaced dental prosthesis either permanently or temporarily fixed within the oral cavity.

By “safe and effective amount”, as used herein, is meant an amount of an agent (e.g., anti-calculus agent) high enough to significantly improve the condition to be treated, but low enough to avoid serious side effects (at a reasonable benefit/risk ratio), within the scope of sound medical/dental judgment. The safe and effective amount of an agent (e.g., anti-calculus agent) may vary with the particular condition being treated, the age and physical condition of the patient being treated, the severity of the condition, the duration of treatment, the nature of concurrent therapy, the specific form of the source employed, and the particular vehicle from which the agent is applied.

By “toothpaste” as used herein is meant paste, powder, and tooth gel formulations unless otherwise specified.

By “oral care composition” or “oral composition” as used herein is meant a product which is not intentionally swallowed for purposes of systemic administration of therapeutic agents, but is retained in the oral cavity for a sufficient time to contact substantially all of the dental surfaces and/or mucosal tissues for purposes of oral activity. In addition, these terms can mean a product which may be intentionally swallowed but not swallowed for the purposes of systemic administration of therapeutic agents. Such oral care compositions include dentifrices, gels, rinses, edible films, and lozenges.

The term “Essential Oil” as used herein is a volatile material derived by a physical process from odorous plant material of a single botanical form and species with which it agrees in name and odor. Essential oils generally constitute the odorous principles of the plants in which they exist. Essential oils are either distilled or expressed. “Distillation” can be water distillation, steam distillation, water and steam distillation or dry distillation. In a water distillation, the plant material is in direct contact with the boiling water. Steam distillation is usually conducted with steam produced in a boiler separate from the still. The steam is blown through the plant material which rests on a grid or trays for quick removal after exhaustion. High pressure steam can be applied if the plant material and its essential oil are sufficiently heat resistant and non-hydrolyzable. Direct and dry distillation can be used for some essential oils. For citrus oils a cold press method can be used to gather the essential oil. The term “Cold Press” as used herein is the method most often used to gather citrus essential oils, predominantly from the peels of citrus fruits. Therefore a citrus oil can also be referred to as a cold-pressed citrus oil. The citrus oils can be further rectified with a distillation process to stabilize the essential oil.

The term “Essential Oil composition” as used herein refers to the inventive blend of essential oils which can ultimately be added to an oral care composition.

The term “Citrus” as used herein includes, but is not limited to, lemon, lime, orange, grapefruit, tangerine, mandarin, Ugli fruit, bergamot, tomato and/or any combination thereof.

The term “Spice” as used herein includes, but is not limited to, cinnamon, cassia, clove, anise, ginger, peppers, nutmeg, allspice, coriander and/or any combination thereof.

The term “Mint” as used herein includes, but is not limited to, spearmint, peppermint, any botanical of the Labiatae family having characteristic square stems and pink, white, or purple verticullate flowers, and/or any combination thereof Carvone, as used herein, can also be a mint.

The term “Rectification” as used herein is a corrective or cleaning process. A rectification is a second distillation of a material without specifications with respect to fractionation or distillation conditions. A steam or vacuum distillation or any second distillation of an essential oil can be considered a rectification. A rectification generally involves a significant loss of material if a high grade fraction is wanted.

By “oral condition or conditions of the oral cavity” as used herein is meant diseases including caries, plaque, breath malodor, gingivitis, and periodontal disease. Oral conditions are further described in WO 02/02096A2, published Jan. 10, 2002, P&G.

By “tooth surfaces” or “teeth surfaces” as used herein is meant the pits, fissures, occlusal surfaces, cleft, crevices, grooves, depressions, interstices, irregularities, inter-proximal surfaces between the teeth and/or along the gum line, the smooth surfaces of teeth, and/or the grinding or biting surfaces of a tooth.

Herein, “comprising” means that other steps and other ingredients which do not affect the end result can be added. This term encompasses the terms “consisting of” and “consisting essentially of”.

By “whole body health” as used herein is meant overall systemic health characterized by a reduction in risk of development of major systemic diseases and conditions including cardiovascular disease, stroke, diabetes, severe respiratory infections, premature births and low birth weights (including postpartum dysfunction in neurologic/development function), and associated increased risk of mortality. It is believed that oral infections could lead to systemic infection. Microbials can spread from the mouth into the bloodstream and other parts of the body, thereby putting a person's health at risk. Oral infection may contribute to the development of a number of serious conditions including heart disease, diabetes, respiratory diseases and premature, underweight births. Whole body health and promotion thereof by treating oral cavity infections is further described in WO 02/02063A2, WO 02/02096A2, WO 02/02128A2, all published Jan 10, 2002.

All percentages and ratios used hereinafter are by weight of total composition, unless otherwise indicated.

All measurements referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

### B. ESSENTIAL OIL COMPOSITION

Compositions of the present invention incorporate the essential oil composition demonstrating antimicrobial properties. In particular, this essential oil composition results in microbial log reductions (also known as bacteria log reductions) of microbial populations Streptococcus sanguis, Streptococcus mutans, and Streptococcus sobrinus. These microbial are pioneer species, the first microbial to absorb, multiply and form micro-colonies; and reduction of these species are correlated to disruption of plaque biofilm. (P. Marsh & M. Martin, Oral Microbiology, 3^{rd} Edition, London, Chapman & Hall Publishers, 1996). These compositions can be used to treat conditions in the oral cavity.

The essential oils can be generated by subjecting botanical materials to a distillation process. The essential oils can undergo additional distillation processes to rectify and purify the essential oils by removing the terpene components via a “head cut” and/or removing the wax components via a “tail cut”. The essential oils can be either synthetic or natural; further a combination of synthetic and natural essential oils can be used. All of the essential oils used herein are commercially available.

The inventive essential oil composition of an oral care composition comprises particular essential oils (described herein as “Group A” and “Group B” essential oils) and/or combinations of the particular essential oils at specified ratios. Combining the essential oils at specified ratios with an oral care carrier, and introducing the resulting oral care composition into the oral cavity has an antimicrobial effect, which can result in the reduction of the microbials correlated to the formation of biofilm; thereby improving the overall health of the oral cavity.

Use of an oral care composition, especially daily use for more than two weeks, comprising the inventive essential oil composition can inhibit the growth of the microbials which are correlated to the formation of biofilm in the oral cavity. It is believed that plaque and eventually tartar and calculus result from a biofilm reaching a more advanced and complex stage of development, trapping additional material and eventually hardening. Therefore, by inhibiting the development of the biofilm, the development of plaque tartar and/or calculus may also be prevented.

The essential oil composition includes essential oils from two groups, Group A and Group B. Combining essential oils from the different groups at the specified ratios can result in the oral benefit.

The impact of the essential oil composition of the present invention on the oral biofilm was measured via microbial cultivation and enumeration, coupled with species characterization of oral lavage expectorates for sampling of oral biofilms on the soft and hard tissues of the oral cavity. After brushing with a dentifrice containing the essential oil composition of the present invention or a control product for one minute, oral lavage samples were collected immediately and at four hours by first, vigorously swishing with ten milliliters of sterile water for one minute and second, spitting the expectorate into a sterile centrifuge tube. The biofilm disruption was measured via enumeration of the expectorate samples and assessment of microbial log reductions, also known as bacterial log reductions, versus water control, of selected microbial populations of Streptococcus sanguis, Streptococcus mutans, and Streptococcus sobrinus. The test were conducted using Sample A—a standard fluoride dentifrice with 1% mint flavor, Sample A′—a standard fluoride dentifrice without flavor, and Sample B—see dentifrice Examples 1 and 2. The results of the overall mean log CFU reduction is as follows:

| Dentifrice | S. sanguis | | S. mutans | | S. sobrinus | |
|---|---|---|---|---|---|---|
| Treatment | Immediate | 4 Hr | Immediate | 4 Hr | Immediate | 4 Hr |
| Sample A | 0.654 | 1.330 | −.0227 | 1.080 | 0.407 | 0.899 |
| Sample A′ | 1.636 | 1.136 | 0.435 | 0.639 | 0.633 | 0.767 |
| Sample B | 2.777 | 3.231 | 1.931 | 1.947 | 2.124 | 1.331 |

Additionally, the overall antimicrobial activity of a dentifrice comprising the inventive oral care composition was evaluated using a broth microdilution MKD method. The broth microdilution MKD method is used to measure the in vitro activity of an antimicrobial agent/formula against a microbial isolate. A sterile plastic tray containing various concentrations of antimicrobial agents/formulas is inoculated with a standard number of test microbials. After overnight incubation at 35° C. or depending upon the growth of each test organism, all of the wells on the plastic tray are subcultured onto nutrient agar plates and incubated. The plates are then examined for growth of the test organism to determine whether microorganisms are still viable or killed by the antimicrobial agent/formula. The MKD is determined by observing the lowest concentration of an antimicrobial agent/formula that demonstrates no growth of the organism after subculture. The results are as follows.

| | S. Mutans | S. Sanguis | P. Gingivalis | F. Nucleatum | E. COLI | C. Albicans |
|---|---|---|---|---|---|---|
| | MKD | MKD | MKD | MKD | MKD | MKD |
| PRODUCT | (1:N) | (1:N) | (1:N) | (1:N) | (1:N) | (1:N) |
| Sample B | 8000 | 8000 | 192 | 384 | 6 | 24 |
| Sample B | 8000 | 6144 | 192 | 384 | 6 | 24 |
| Sample A | 2048 | 2048 | 192 | 192 | 6 | 24 |
| Sample A′ | 1536 | 768 | 192 | 1024 | 6 | 24 |

Both dentifrices comprising the inventive essential oil composition have an equal or higher MKD for each of the tested microbials than dentifrices having standard flavor formulas, both with and without flavor. As shown by the above results table, the sum of the MKDs for both dentifrices comprising the inventive oral care composition are higher than the sum of MKDs for the standard dentifrice having 1% flavor oils. Additionally, a standard dentifrice having no flavor oil component is tested.

In one embodiment the essential oil composition comprises a ratio of Group A essential oils to Group B essential oils of from about 2:1 to about 10:1. In another embodiment the Group A essential oils to Group B essential oils is from about 2:1 to about 6:1. In another embodiment the Group A essential oils to Group B essential oils is from about 3:1 to about 5:1. The essential oil composition can be added to an oral care composition in an amount of from at least about 1% by weight of the oral care composition. In one embodiment the essential oil composition is added to the oral care composition at a level of from about 1% to about 5% by weight of the oral care composition. In another embodiment theessential oil composition is added to the oral care composition at from about 1% to about 3% by weight of the oral care composition. In another embodiment the essential oil composition is added to the oral care composition at from about 1.5% to about 2.5%, and in yet another embodiment the essential oil composition is added to the oral care composition at from about 1.5 to about 2% by weight of the oral care composition.

1. Group A Essential Oils

The essential oils of Group A include mint and/or spice essential oils. The mint essential oils include, but are not limited to, spearmint, peppermint, any botanical of the Labiatae family having characteristic square stems and pink, white, or purple verticullate flowers, and/or any combination thereof. Additionally, carvone can be combined with any of the aforementioned mint essential oils. Carvone is the primary constituent of spearmint oil. Carvone can be either natural or synthetic. Synthetic carvone can be prepared from d-limonene, which is a monoterpene derived from the citrus oils. The spice essential oils include, but are not limited to, cinnamon, cassia, clove, anise, ginger, pepper, nutmeg, allspice, coriander and/or any combination thereof.

In one embodiment the mint essential oil is spearmint. Spearmint oil can be produced by steam distillation from the flowering tops of the mentha viridis plant, which is partially dried prior to the distillation. In one embodiment the spice essential oil is cinnamon, and in another embodiment the spice essential oil is cassia. The cassia oil, or Chinese cinnamon oil, can be steam distilled from the leaves of cinnamomum cassia; whereas cinnamon bark oil can be steam distilled from the dried inner bark of the shoots of cinnamomum zeylanicum, and cinnamon leaf oil can be steam distilled from dried leaves and twigs of the same tree that yields the cinnamon bark oil.

In one embodiment the essential oils composition comprises at least two essential oils from Group A. In another embodiment the essential oil composition comprises at least three essential oils from Group A. In yet another embodiment the essential oil composition comprises one mint and one spice essential oil.

In one embodiment a combination of a mint essential oil and a spice essential oil from Group A are provided in a ratio of from about 10:1 to about 4:1. In another embodiment a mint essential oil and a spice essential oil from Group A are provided in a ratio of from about 9:1 to about 5:1. In another embodiment a mint essential oil and a spice essential oil from Group A are provided in a ratio of from about 8:1 to about 6:1. In yet another embodiment a combination of spearmint essential oil and cassia or cinnamon essential oil from Group A are provided in a ratio of from about 8.5:1 to about 5.5:1. In yet another embodiment a combination of spearmint essential oil to carvone essential oil to cinnamon or cassia essential oil from Group A is from about 8:4:1 to about 3:1:1.

In one embodiment the Group A essential oils can be added to the oral care composition in an amount of from about 0.5% to about 4.5% by weight of the oral care composition. In another embodiment Group A essential oils can be added to the oral care composition in an amount of from about 0.5% to about 2.5% by weight of the oral care composition. In another embodiment the Group A essential oils can be added to the oral care composition in an amount of from about 0.75% to about 3.75%, and another embodiment from about 0.75% to about 2%, and in yet another embodiment in an amount of from about 0.75% to about 1.5% by weight of the oral care composition.

2. Group B Essential Oils

The essential oils of Group B include citrus essential oils. The citrus oils include, but are not limited to, lemon, lime, orange, grapefruit, tangerine, mandarin, Ugli fruit, bergamot, tomato and/or any combination thereof. Traditionally, the citrus essential oil is produced from the peel of the fruit. In one embodiment the Group B essential oil is orange oil. In another embodiment the Group B essential oil is lemon oil. In yet another embodiment the Group B essential oil is a combination of orange and lemon oils at a ratio of from about 1.5:1 to about 3.5:1. In yet another embodiment the Group B essential oil, is a combination of orange and lemon oils at a ratio of about 2.5:1 to about 2:1. Group B essential oils can be added to oral care compositions in an amount of from about 0.10 to about 1.7% by weight of the oral care composition. In another embodiment the Group B essential oils can be added to the oral care composition in an amount of from about 0.10% to about 1.25%, and in another embodiment in an amount of from about 0.20% to about 1.25%, and in another embodiment in an amount of from about 0.35 to about 0.5% by weight of the oral care composition.

### C. OPTIONAL INGREDIENTS

1. Oral Care Carriers

The oral care composition comprising the combination of Group A and B essential oils can also comprise a variety of oral care carriers including dentifrice, rinse, gel, gum, candy, confectionary or lozenge, edible film, and/or a quick dissolving wafer such as described in U.S. Pat. No. 6,221,392. Additionally, the oral care composition can be disposed on a dental wipe such as described in U.S. Pat. No. 6,721,987 and WO Application No. 02/069753.

The choice of a carrier to be used is determined by the way the composition is to be introduced into the oral cavity. If a tooth paste (including tooth gels, chewable tablets etc.) is to be used, then a “toothpaste carrier” is chosen as disclosed in, e.g., U.S. Pat. No. 3,988,433, (e.g., abrasive materials, sudsing agents, binders, humectants, flavoring and sweetening agents, etc.). If a mouth rinse is to be used, then a “mouth rinse carrier” is chosen, as disclosed in, e.g., U.S. Pat. No. 3,988,433 (e.g., water, flavoring and sweetening agents, etc.). Similarly, if a mouth spray is to be used, then a “mouth spray carrier” is chosen. If a sachet is to be used, then a “sachet carrier” is chosen (e.g., sachet bags flavoring and sweetening agents). If a subgingival gel is to be used (for delivery of actives into the periodontal pockets or around the periodontal pockets), then a “subgingival gel carrier” is chosen as disclosed in, e.g. U.S. Pat. Nos. 5,198,220; and 5,242,910. Carriers suitable for the preparation of compositions of the present invention are well known in the art. Their selection will depend on secondary considerations like taste, cost, and shelf stability, etc.

In one embodiment of the subject invention, the compositions are in the form of dentifrices, such as toothpastes, tooth gels and tooth powders. Components of such toothpaste and tooth gels may include one or more of a dental abrasive (from about 10% to about 50%), a surfactant (from about 0.5% to about 10%), a thickening agent (from about 0.1% to about 5%), a humectant (from about 10% to about 55%), an essential oil component (from about 0.04% to about 2%), a sweetening agent (from about 0.1% to about 3%), a coloring agent (from about 0.01% to about 0.5%) and water (from about 2% to about 4 5%). Such toothpaste or tooth gel may also include one or more of an additional anticaries agent (from about 0.05% to about 10% additional anticaries agent), and an anticalculus agent (from about 0.1% to about 13%). The tooth powders, of course, contain substantially all non-liquid components.

Other embodiments of the compositions of the subject invention are mouthwashes, including mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an of additional anticaries agent (from about 0.05% to about of additional anticaries agent), and an anticalculus agent (from about 0.1% to about 13%).

Other embodiments of the compositions of the subject invention are dental solutions. Components of such dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Another embodiment of the present invention includes a candy, confection and/or lozenge. The carrier material for the candy, confection and/or lozenge can be chosen from chewable or non chewable materials. The chewable material can be selected from gums including, but not limited to, agar gum, and gelatine; low boiled sugar candy base and gum base materials. Hard and low boiled candy carrier, pressed tablets and the like can comprise greater than about 70% bulk sweetener including suitable sugar and sugar syrups including cariogenic and non-cariogenic materials. Low boiled candies can also comprise butter to form chewable toffee. For jelly and gum drop compositions the carrier can comprise greater than about 25% bulk sweetener and additionally comprise gums including gum arabic, gelatine, agar powder and the like. In addition to the essential oil composition, the candy, confections and/or lozenge can additionally comprise other oral and/or therapeutic actives.

Another embodiment of the present invention includes chewing gum compositions. Such compositions may be in the form of a conventional chewing gum or any other product form which is suitable for chewing. Suitable physical forms include sticks, dragees, chiclets, and batons. A chewing gum is typically retained in the oral cavity for a time sufficient to allow ingredients released to contact substantially all of the dental surfaces and/or oral tissues for purposes of oral activity. Chewing gums can comprise abrasive polishing materials, elastomers, resins, plasticizers, fats, solvents, bulking agents, sweeteners, absorbents, orally active metallic ions, cationic material, fluoride ion sources, additional anticalculus agents, antimicrobial agents, buffers, whitening agents, alkali metal bicarbonate salts, thickening materials, humectants, water, surfactants, titanium dioxide, flavoring agents, xylitol, coloring agents, and mixtures thereof.

In another embodimeent of the present invention, the essential oil composition is present in an edible film that is physiologically acceptable and particularly well adapted to adhere to and dissolve in the oral cavity of a consumer. Examples of suitable edible film and methods for making such films are described in U.S. Pat. Nos. 6,596,298; 5,733,584; 5,948,430; 6,177,096;, and W.O. Application No. 00/18365. An edible film comprising pullulan can be used. The edible films of the present invention can be used to deliver or release the essential oil composition and/or other oral care active. The edible films can include a variety of other suitable ingredients such as softeners, colorants, flavoring agents, emulsifiers, surfactants, thickening agents, binding agents, sweeteners, fragrances, and combinations thereof.

The compositions of the present invention can further comprise abrasives, surfactants, thickening agents, humectants and flavorants previously disclosed it the art.

2. Abrasives

A dentifrice comprising the inventive essential oil composition may comprise dental abrasives. However, an oral care composition, such as a dentifrice, can be essentially free of abrasives. The material selected is to be compatible within the composition of interest and does not excessively abrade dentin. Suitable abrasives include, for example silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Mixtures of abrasives may also be used.

Silica dental abrasives of various types can be used because of their unique benefits of exceptional dental cleaning and polishing performance without unduly abrading tooth enamel or dentine. The silica abrasive polishing materials herein, as well as other abrasives, can have an average particle size ranging between about 0.1 to about 30 microns, and in another embodiment from about 5 to about 15 microns. The abrasive can be precipitated silica or silica gels such as the silica xerogels described in U.S. Pat. No. 3,538,230, and U.S. Pat. No. 3,862,307. Suitable silica xerogels are the silica xerogels marketed under the trade name “Syloid” by the W.R. Grace & Company, Davison Chemical Division. Also precipitated silica materials, such as those marketed by the J. M.

Huber Corporation under the trade name, Zeodent®, can be used; in one embodiment the silica carrying the designation Zeodent 119® can be used. The types of silica dental abrasives useful in the toothpastes of the present invention are described in more detail in U.S. Pat. No. 4,340,583. The abrasive in the toothpaste compositions described herein is generally present at a level of from about 6% to about 70% by weight of the composition. The toothpastes can contain from about 10% to about 50% of abrasive, by weight of the composition. Mixtures of the abrasives can be used. Examples of suitable precipitated silica is the silica disclosed in U.S. Pat. Nos. 5,603,920; 5,589,160; 5,658,553;

5,651,958. Additionally, solutions such as mouth spray, and mouthwash typically contain no abrasive.

3. Sudsing Agents (Surfactants)

Suitable sudsing agents are those which are reasonably stable and form foam throughout a wide pH range. Sudsing agents include nonionic, anionic, amphoteric, cationic, zwitterionic, synthetic detergents, and mixtures-thereof Many suitable-nonionic and amphoteric surfactants are disclosed in U.S. Pat. Nos. 3,988,433; and 4,051,234, and many suitable nonionic surfactants are disclosed in U.S. Pat. No. 3,959,458.

4. Thickening Agents

In preparing toothpaste or gels, it is necessary to add some thickening material to provide a desirable consistency of the composition, to provide desirable release characteristics upon use, to provide shelf stability, and to provide stability of the composition, etc. Suitable thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Thickening agents can include however, except polymeric polyether compounds, e.g., polyethylene or polypropylene oxide (M.W. 300 to 1,000,000), capped with alkyl or acyl groups containing 1 to about 18 carbon atoms.

A suitable class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol® series. Particularly the carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. Nos. 5,198,220; 5,242,910; and 4,443,430.

Thickening agents in an amount from about 0.1% to about 15%, or from about 0.2% to about 6%, in another embodiment from about 0.4% to about 5%, by weight of the total toothpaste or gel composition, can be used. Higher concentrations can be used for sachets, non-abrasive gels and subgingival gels.

5.Humectants

Another optional component of the topical, oral-carriers of the compositions of the subject invention is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, can comprise from about 0% to about 70%, and in another embodiment from about 5% to about 25%, by weight of the compositions herein. Suitable humectants for use in compositions of the subject invention include edible polyhydric alcohols such as glycerin, sorbitol, xylitol, butylene glycol, polyethylene glycol, and propylene glycol, especially sorbitol and glycerin.

6. Sweetening Agents

Sweetening agents which can be used include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A composition can contain from about 0.1% to about 10% of these agents, in another embodiment from about 0.1% to about 1I%, by weight of the composition.

In addition to sweetening agents, coolants, salivating agents, warming agents, and numbing agents can be used as optional ingredients in compositions of the present invention. These agents are present in the compositions at a level of from about 0.001% to about 10%, in another embodiment from about 0.1% to about 1%, by weight of the composition.

The coolant can be any of a wide variety of materials. Included among such materials are carboxamides, menthol, ketals, diols, and mixtures thereof. Suitable coolants in the present compositions include the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro and a levorotatory isomers of these compounds and racemic mixtures thereof. TK10 is described in U.S. Pat. No. 4,459,425. WS-3 and other agents are described in U.S. Pat. No. 4,136,163.

Suitable salivating agents of the present invention include Jambu® manufactured by Takasago. Suitable warming agents include capsicum and nicotinate esters, such as benzyl nicotinate. Suitable numbing agents include benzocaine, lidocaine, clove bud oil, and ethanol.

7. Cosmetic or Therapeutic Actives

The oral care composition may also comprise suitable cosmetic and/or therapeutic actives. Such actives include any material that is generally considered safe for use in the oral cavity and that provides changes to the overall appearance and/or health of the oral cavity, including, but not limited to, anti-calculus agents, fluoride ion sources, stannous ion sources, whitening agents, anti-microbial, anti-plaque agents, anti-inflammatory agents, nutrients, antioxidants, anti-viral agents, analgesic and anesthetic agents, H-2 antagonists, components which impart a clean feel to the teeth, pigments and colorants, fragrances and sensates, and mixture thereof. When present, the level of cosmetic and/or therapeutic active in the composition is, in one embodiment from about 0.001% to about 90%, in another embodiment from about 0.01% to about 50%, and in another embodiment from about 0. 1% to about 30%, by weight of the composition.

The following is a non-limiting list of actives that may be used in the present invention.

a. Anticalculus Agent

Compositions of the present invention may also comprise an anti-calculus agent, which in one embodiment may be present from about 0.05% to about 50%, by weight of the oral care composition, in another embodiment is from about 0.05% to about 25%, and in another embodiment is from about 0.1% to about 15%. The anti-calculus agent may be selected from the group consisting of polyphosphates (including pyrophosphates) and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof, polyolefin phosphonates and salts thereof; polypeptides; and mixtures thereof. In one embodiment, the salts are alkali metal salts. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation, which are commercially known as Sodaphos (n≈6), Hexaphos (n≈13), and Glass H (n≈21, sodium hexametaphosphate), and mixtures thereof. The pyrophosphate salts useful in the present invention include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophospbate, tetrasodium pyrophosphate (Na₄P₂O₇), tetrapotassium pyrophosphate (K₄P₂O₇), and mixtures thereof. Polyolefin sulfonates include those wherein the olefin group contains 2 or more carbon atoms, and salts thereof. Polyolefin phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof, azacyclohexane-2,2-diphosphonic acid, azacyclopentane-2,2-diphosphonic acid, N-methyl-azacyclopentane-2,3-diphosphonic acid, EHDP (ethane-1-hydroxy-1,1,-diphosphonic acid), AHP (azacycloheptane-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefin group contains 2 or more carbon atoms. Polypeptides include polyaspartic and polyglutamic acids.

b. Stannous Ion

The oral care compositions of the present invention may include a stannous ion source. The stannous ions may be provided from stannous fluoride and/or other stannous salts. Stannous fluoride has been found to help in the reduction of gingivitis, plaque, sensitivity, and in improved breath benefits. The stannous ions provided in an oral composition will provide efficacy to a subject using the composition. Although efficacy could include benefits other than the reduction in gingivitis, efficacy is defined as a noticeable amount of reduction in in situ plaque metabolism. Formulations providing such efficacy typically include stannous levels provided by stannous fluoride and/or other stannous salts ranging from about 3,000 ppm to about 15,000 ppm stannous ions in the total composition. The stannous ion is present in an amount of from about 4,000 ppm to about 12,000 ppm, in one embodiment from about 5,000 ppm to about 10,000 ppm. Other stannous salts include organic stannous carboxylates, such as stannous acetate, stannous gluconate, stannous oxalate, stannous malonate, stannous citrate, stannous ethylene glycoxide, stannous formate stannous sulfate, stannous lactate, stannous tartrate, and the like. Other stannous ion sources include, stannous halides such as stannous chlorides, stannous bromide, stannous iodide and stannous chloride dihydride. In one embodiment the stannous ion source is stannous fluoride in another embodiment, stannous chloride dihydrate. The combined stannous salts may be present in an amount of from about 0.001% to about 11%, by weight of the compositions. The stannous salts may, in one embodiment, be present in an amount of from about 0.01% to about 7%, in another embodiment from about 0.1% to about 5%, and in another embodiment from about 1.5% to about 3%, by weight of the composition.

c. Whitening Agent

A whitening agent may be included as an active in the present compositions. The actives suitable for whitening are selected from the group consisting of alkali metal and alkaline earth metal peroxides, metal chlorites, perborates inclusive of mono and tetrahydrates, perphoshates, percarbonates, peroxyacids, and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures thereof. In one embodiment the peroxide compound is carbamide peroxide. Suitable metal chlorites include calcium chlorite, barium chlorite, magnesium chlorite, lithium chlorite, sodium chlorite, and potassium chlorite. Additional whitening actives may be hypochlorite and chlorine dioxide. In one embodiment the chlorite is sodium chlorite. In another embodiment the percarbonate is sodium percarbonate. In one embodiment the persulfates are oxones. The level of these substances is dependent on the available oxygen or chlorine, respectively, that the molecule is capable of providing to bleach the stain. In one embodiment the whitening agents may be present at levels from about 0.01% to about 40%, in another embodiment from about 0.1% to about 20%, in another embodiment form about 0.5% to about 10%, and in another embodiment from about 4% to about 7%, by weight of the composition.

d. Anti-Microbial Agent

Anti-microbial agents may be included in the compositions of the present invention. Such agents may include, but are not limited to: 5-chloro-2-(2,4-dichlorophenoxy)-phenol, comnuonly referred to as triclosan; 8-hydroxyquinoline and its salts; copper II compounds, including, but not limited to, copper(II) chloride, copper(II) sulfate, copper(II) acetate, copper(II) fluoride and copper(II) hydroxide; phthalic acid and its salts including, but not limited to those disclosed in U.S. Pat. No. 4,994,262, including magnesium monopotassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; iodine; sulfonamides; bisbiguanides; phenolics; delmopinol, octapinol, and other piperidino derivatives; niacin preparations; zinc or stannous ion agents; nystatin; grapefruit extract; apple extract; thyme oil; thymol; antibiotics such as augmentin, amoxicillin, tetracycline, doxycycline, minocycline, metronidazole, neomycin, kanamycin, cetylpyridinium chloride, and clindamycin; analogs and salts of the above; methyl salicylate; hydrogen peroxide; metal salts of chlorite; and mixtures of all of the above. Anti-microbial components may be present from about 0.001% to about 20% by weight of the composition.

e. Anti-Plaque Agent

The compositions of the present invention may include an anti-plaque agent such as stannous salts, copper salts, strontium salts, magnesium salts or a dimethicone copolyol. The dimethicone copolyol is selected from C12 to C20 alkyl dirnethicone copolyols and mixtures thereof. In one embodiment the dimethicone copolyol is cetyl dimethicone copolyol marketed under the Trade Name Abil EM90. The dimethicone copolyol in one embodiment can be present in a level of from about 0.001% to about 25%, in another embodiment from about 0.01% to about 5%, and in another embodiment from about 0. I% to about 1.5% by weight of the composition.

f. Anti-Inflammatory Agent

Anti-inflammatory agents can also be present in the oral care compositions of the present invention. Such agents may include, but are not limited to, non-steroidal anti-inflammatory (NSAID) agents oxicams, salicylates, propionic acids, acetic acids and fenamates. Such NSAIDs include but are not limited to ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone and acetaminophen. Use of NSAIDs such as ketorolac are claimed in U.S. Pat. No. 5,626,838. Disclosed therein are methods of preventing and/or treating primary and reoccurring squamous cell carcinoma of the oral cavity or oropharynx by topical administration to the oral cavity or oropharynx of an effective amount of an NSAID. Suitable steroidal anti-inflammatory agents include corticosteroids, such as fluccinolone, and hydrocortisone.

g. Nutrients

Nutrients may improve the condition of the oral cavity and can be included in the oral care compositions of the present invention. Nutrients include minerals, vitamins, oral nutritional supplements,. enteral nutritional supplements, and mixtures thereof. Useful minerals include calcium, phosphorus, zinc, manganese, potassium and mixtures thereof. Vitamins can be included with minerals or used independently. Suitable vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but are not limited to L-Tryptophan, L-Lysine, Methionine, Threonine, Levocarnitine or L-camitine and mixtures thereof. Lipotropics include, but are not limited to, choline, inositol, betaine, linoleic acid, linolenic acid, and mixtures thereof. Fish oil contains large amounts of Omega-3 (N-3) polyunsaturated fatty acids, eicosapentaenoic acid and docosahexaenoic acid. Enteral nutritional supplements include, but are not limited to, protein products, glucose polymers, corn oil, safflower oil, medium chain triglycerides. Minerals, vitamins, oral nutritional supplements and enteral nutritional supplements are described in more detail in Drug Facts and Comparisons (loose leaf drug information service), Wolters Kluer Company, St. Louis, Mo., ©1997, pps. 3-17 and 54-57.

h. Antioxidants

Antioxidants are generally recognized as useful in oral care compositions. Antioxidants are disclosed in texts such as Cadenas and Packer, The Handbook of Antioxidants, © 1996 by Marcel Dekker, Inc. Antioxidants useful in the present invention include, but are not limited to, Vitamin E, ascorbic acid, Uric acid, carotenoids, Vitamin A, flavonoids and polyphenols, herbal antioxidants, melatonin, aminoindoles, lipoic acids and mixtures thereof.

i. Analgesic and Anesthetic Agents

Anti-pain or desensitizing agents can also be present in the oral care compositions of the present invention. Analgesics are agents that relieve pain by acting centrally to elevate pain threshold without disturbing consciousness or altering other sensory modalities. Such agents may include, but are not limited to: strontium chloride, potassium nitrate; sodium fluoride; sodium nitrate; acetanilide; phenacetin; acertophan; thiorphan; spiradoline; aspirin; codeine; thebaine; levorphenol; hydroimorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 7²9-737.

j. H-1 and H-2 Antagonists

The present invention may also optionally comprise selective H-1 and H-2 antagonists including compounds disclosed in U.S. Pat. No. 5,294,433.

k. Pigments and Colorants

Pigments may be added to the compositions herein to more precisely indicate the locations at which the composition has actually been in contact. Additionally, these substances may be suitable for modifying the color of the teeth to satisfy the consumer. These substances comprise particles that when applied on the tooth surface modify that surface in terms of absorption and, or reflection of light. Such particles provide an appearance benefit when a film containing such particles is applied over the surfaces of a tooth or teeth. Pigments, dyes, colorants and lakes may also be added to modify the appearance of the compositions herein to render the product more acceptable to the consumer. Appropriate pigment levels are selected for the particular impact that is desirable to the consumer. For example, for teeth that are particularly dark or stained one would typically use pigments in sufficient amounts to lighten the teeth. On the other hand, where individual teeth or spots on the teeth are lighter than other teeth, pigments to darken the teeth may be useful. The levels of pigments and colorants may be in the range of about 0.001% to about 20%, in one embodiment from about 0.01% to about 15% and in another embodiment from about 0.1% to about 10% by total weight of the chewing oral care composition.

Pigments and colorants include inorganic white pigments, inorganic colored pigments, pearling agents, filler powders and the like; see Japanese Published Patent Application Kokai No. 9 [1997]-100215, published Apr. 15, 1997. Specific examples are selected from the group consisting of talc, mica, magnesium carbonate, calcium carbonate, magnesium silicate, aluminum magnesium silicate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, silk powder, crystalline cellulose, starch, titanated mica, iron oxide titanated mica, bismuth oxychloride, and mixtures thereof. In one embodiment the pigments and colorants are those selected from the group consisting of titanium dioxide, bismuth oxychloride, zinc oxide, Opatint D&C Red 27, CI 16185:1 Acid 27 Lake E123, CI14720:1 Carmosoisine Aluminum Lake E122, Red 7 Lake, or Red 30 Lake and mixtures thereof.

i. Additional Actives

Additional actives suitable for use in the present invention may include, but are not limited to, insulin, steroids, herbal and other plant derived remedies, and anti-neoplastics. Additionally, anti-gingivitis or gum care agents known in the art may also be included. Components which impart a clean feel to the teeth may optionally be included. These components may include, for example, baking soda or Glass-H. Also, it is recognized that in certain forms of therapy, combinations of these above-named agents may be useful in order to obtain an optimal effect. Thus, for example, an anti-microbial and an anti-inflammatory agent may be combined in a single oral care composition to provide combined effectiveness.

Optional agents to be used include such known materials as synthetic anionic polymers, including polyacrylates and copolymers of maleic anhydride or acid and methyl vinyl ether (e.g., Gantrez), as described, for example, in U.S. Pat. No. 4,627,977, as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Additionally, the oral care composition can include a polymer carrier, such as those described in U.S. Pat. Nos. 6,682,722 and 6,589,512 and U.S. application Ser. Nos. 10/424,640 and 10/430,617.

m. Alkali Metal Bicarbonate Salt

The present invention may also include an alkali metal bicarbonate salt. Alkali metal bicarbonate salts are soluble in water and unless stabilized, tend to release carbon dioxide in an aqueous system. In one embodiment sodium bicarbonate, also known as baking soda, is the alkali metal bicarbonate salt. The present composition may contain from about 0.5% to about 30%, in another embodiment from about 0.5% to about 1⁵%/o, and in another embodiment from about 0.5% to about 5% of an alkali metal bicarbonate salt.

n. Respiratory Actives

Also useful herein are miscellaneous respiratory agents selected from the group consisting of leukotriene receptor antagonists such as zafirlukast, zileuton; nasal inhalant products such as corticosteroids, other steroids, beclomethasone, flunisolide, triamcinolone; mucolytics such as acetylcysteine; anticholinergics such as ipratropium bromide; cromolyn sodium, nedocromil sodium; lung surfactants; and mixtures thereof.

Preferably these agents are present in the compositions at a level of from about 0.001% to about 10%, preferably from about 0.1% to about 5%, by weight of the composition.

8. Miscellaneous Carriers

Water employed in the preparation of commercially suitable oral compositions should be of low ion content and free of organic impurities. Water generally comprises from about 5% to about 70%, and in another embodiment from about 20% to about 50%, by weight of the composition herein. These amounts of water include the free water which is added plus that which is introduced with other materials, such as with sorbitol.

Titanium dioxide may also be added to the present cobmposition. Titanium dioxide is a white powder which adds opacity to the compositions. Titanium dioxide generally comprises from about 0.25% to about 5% by weight of the dentifrice compositions.

Antimicrobial antiplaque agents may also be optionally present in oral compositions. Such agents may include, but are not limited to, triclosan, 5-chloro-2-(2,4-dichlorophenoxy)-phenol, as described in The Merck Index, 11^{th} ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. ⁴624); sanguinarine (Merck Index, no. 8320); benzalkonium chloride (Merck Index, no. 1066); salicylanilide (Merck Index, no. 8299); domiphen bromide (Merck Index, no. 34.11); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minbcycline, and metronidazole; and analogs and salts of the above antimicrobial antiplaque agents. If present, the antimicrobial antiplaque. agents generally comprise from about 0.1% to about -5% by-weight-of the-compositions of the present invention.

Other optional agents include synthetic anionic polymeric polycarboxylates being employed in the form of their free acids or partially or fully neutralized water soluble alkali metal (e.g. potassium and sodium) or ammonium salts and are disclosed in U.S. Pat. Nos. 4,152,420; 3,956,480; 4,138,477; 4,183,914; and 4,906,456. In one embodiment the copolymers can be 1:4 to 4:1 copolymers of maleic anhydride or acid with another polymerizable ethylenically unsaturated monomer, in particular the methyl vinyl ether (methoxyethylene) can have a molecular weight (M.W.) of about 30,000 to about 1,000,000. These copolymers are available for example as Gantrez (AN 139 (M.W. 500,000), A.N. 119 (M.W. 250,000) and as S-97 Pharmaceutical Grade (M.W. 70,000), of GAF Corporation.

### C. METHODS OF USE

A safe and effective amount of the compositions, of the present invention may be topically applied to the mucosal tissue of the oral cavity, to the gingival tissue of the oral cavity, and/or to the surface of the teeth, for the treatment or prevention of the above mentioned conditions of the oral cavity, in several conventional ways. For example, the gingival or mucosal tissue may be rinsed with a solution (e.g., mouth rinse, mouth spray); or in a dentifrice (e.g., toothpaste, tooth gel, chewable tablet, tooth powder), the gingival/mucosal tissue and/or teeth are bathed in the liquid and/or lather generated by brushing the teeth. A chewable dentifrice tablet can be chewed thus delivering the tooth actives to the surfaces of the oral cavity such as described in U.S. patent application Publication Nos. 2004/0101493 and 2004/0101494. The teeth can then be brushed after the tablet is chewed. Other non-limiting examples include applying a non-abrasive gelpor paste, directly to the gingival/mucosal tissue or to the teeth with or without an oral care appliance described below.

For the method of treating diseases or conditions of the oral cavity, including caries, a safe and effective amount of the present compositions can be applied to the gingival/mucosal tissue and/or the teeth (for example, by rinsing with a mouthrinse, directly applying a non-abrasive gel with or without a device, applying a dentifrice or a tooth gel with a toothbrush, etc.) of a person in need thereof, for at least about 10 seconds, in another embodiment from about 20 seconds to about 10 minutes, in even another embodiment from about 30 seconds to about 60 seconds. The method often involves expectoration of most of the composition following such contact. The frequency of such contact can be from about once per week to about four times per day, in another embodiment from about thrice per week to about three times per day, and in another embodiment from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. If delivery to the periodontal pockets is desirable, a mouthrinse can be delivered to the periodontal pocket using a syringe or water injection device. These devices are known to one skilled in the art. Devices of this type include “Water Pik” by Teledyne Corporation. After irrigating, the subject can swish the rinse in the mouth to also cover the dorsal tongue and other gingival and mucosal surfaces. In addition a toothpaste, non-abrasive gel, toothgel, etc. can be brushed onto the tongue. surface and other gingival and mucosal tissues of the oral cavity. The period of such treatment typically ranges from about one day to a lifetime. The subject may repeat the application as needed. The duration of treatment can be from about 3 weeks to about 3 months, but may be shorter or longer depending on the severity of the condition being treated, the particular delivery form utilized and the patient's response to treatment.

For a method of reducing the microbials correlated to the formation of biofilm in the oral cavity, a safe and effective amount of the oral care composition of the present invention can be administered to the oral cavity of a subject in need thereof.

Additionally, the oral cavity can be treated by using edible films, candy, confectionary, lozenges and/or gums. Such compositions are retained in the oral cavity for a substantial period of time during consumption, and therefore, provide an ideal product form for a portable oral care product.

The compositions of this invention are useful for both human and other lower animal (e.g. pets, zoo, or domestic animals) applications.

The following non-limiting examples further describe embodiments within the scope of the present invention. Many variations of these examples are possible without departing from the scope of the invention.

### D. EXAMPLESs

1. Dentifrice

The following dentifrice compositions are made by conventional processing and are described below:

| | EXP 1 | EXP 2 | EXP 3 | EXP 4 | EXP 5 | EXP 6 | EXP 7 |
|---|---|---|---|---|---|---|---|
| INGREDIENT | Wt/Wt % | Wt/Wt % | Wt/Wt % | Wt/Wt % | Wt/Wt % | Wt/Wt % | Wt/Wt % |
| Saccharin | 0.320 | 0.320 | 0.320 | 0.320 | 0.320 | 0.320 | 0.320 |
| Sodium USP (a) | | | | | | | |
| Trisodium | 1.450 | 1.450 | 1.450 | 1.450 | 1.450 | 1.450 | 1.450 |
| Phosphate | | | | | | | |
| Xanthan Gum NF | 0.475 | 0.475 | 0.475 | 0.475 | 0.475 | 0.475 | 0.475 |
| Sodium Fluoride | 0.243 | 0.243 | 0.243 | 0.243 | 0.243 | 0.243 | 0.243 |
| USP | | | | | | | |
| Carbomer 956 | 0.300 | 0.300 | 0.300 | 0.300 | 0.300 | 0.300 | 0.300 |
| Monosodium | 0.590 | 0.590 | 0.590 | 0.590 | 0.590 | 0.590 | 0.590 |
| Phosphate | | | | | | | |
| Sorbitol Soln | 61.942 | 62.242 | 62.442 | 61.942 | 62.242 | 62.442 | 62.242 |
| USP (70%) (b) | | | | | | | |
| Silica Abrasive | 20.000 | 20.000 | 20.000 | 20.000 | 20.000 | 20.000 | 20.000 |
| USP | | | | | | | |
| Purified Water, | 8.980 | 8.980 | 8.980 | 8.980 | 8.980 | 8.980 | 8.980 |
| USP (b) | | | | | | | |
| Sodium Lauryl | 4.000 | 4.000 | 4.000 | 4.000 | 4.000 | 4.000 | 4.000 |
| Sulfate 28% Soln | | | | | | | |
| Spearmint - | 0.63 | 0.49 | 0.40 | x | 0.49 | 0.40 | 0.40 |
| natural | | | | | | | |
| Spearmint - | x | x | x | 0.63 | x | x | x |
| Synthetic | | | | | | | |
| 1-carvone | 0.22 | 0.18 | 0.15 | 0.22 | 0.18 | 0.15 | 0.18 |
| Cassia - Natural | x | x | 0.10 | x | x | 0.10 | x |
| Cassia- synthetic | 0.10 | 0.10 | x | 0.10 | x | x | x |
| Cinnamon | x | x | x | x | 0.10 | x | 0.10 |
| Orange 10x | 0.17 | 0.13 | 0.10 | 0.17 | x | 0.10 | x |
| Lemon 10x | 0.08 | 0.06 | 0.05 | 0.08 | 0.06 | 0.05 | 0.06 |
| Grapefruit 10x | x | x | x | x | 0.13 | x | 0.13 |
| Menthol | 0.30 | 0.24 | 0.20 | 0.30 | 0.24 | 0.20 | x |
| WS-3 | x | x | x | x | x | x | 0.33 |
| Dye, FD&C Blue | 0.200 | 0.200 | 0.200 | 0.200 | 0.200 | 0.200 | 0.200 |
| No. 1 Soln (c) | | | | | | | |
| Total | 100.00 | 100.00 | 100.000 | 100.00 | 100.00 | 100.00 | 100.00 |

Chewable Dentifrice Tablet

The following chewable compressed tablets, containing sodium fluoride, are made by conventional tableting processing techniques by mixing the following:

| | EXP 1 | EXP 2 | EXP 3 | EXP 4 | EXP 5 |
|---|---|---|---|---|---|
| Material | % w/w | % w/w | % w/w | % w/w | % w/w |
| Na Fluoride | 0.243 | 0.0884 | 0.0552 | 0.11 | 0.11 |
| Na Lauryl Sulfate | 1.5 | x | x | 1.5 | 1.5 |
| Poloxamer 407 | x | 7.5 | x | X | x |
| PEG 40 Sorbitan | x | x | 2 | X | x |
| Di-iso Stearate | | | | | |
| Silica | 20 | x | x | 20 | 20 |
| Ca Pyrophosphate | x | 40 | x | X | x |
| Dicalcium | x | x | 40 | X | x |
| Phosphate | | | | | |
| Tetra Sodium | x | 5 | 5 | X | 5 |
| Pyrophosphate | | | | | |
| Na Saccharin | 0.5 | x | 0.4 | 0.4 | 0.4 |
| Acesulfame K | x | .3 | x | X | x |
| Sucralose | x | x | 0.1 | X | x |
| Aspartame | x | .3 | x | X | x |
| Spearmint (natural) | 0.56 | 0.63 | x | 0.67 | 0.61 |
| Spearmint | x | x | 0.67 | X | x |
| (synthetic) | | | | | |
| 1-carvone | 0.29 | 0.22 | 0.18 | 0.20 | 0.29 |
| Cassia- (synthetic) | 0.10 | 0.10 | 0.10 | 0.08 | x |
| Cassia (natural) | x | x | x | X | 0.05 |
| Orange 10x | 0.15 | 0.17 | 0.18 | 0.10 | 0.20 |
| Lemon 10x | 0.10 | 0.08 | 0.12 | 0.20 | 0.10 |
| Menthol | 0.30 | 0.30 | 0.25 | 0.25 | 0.25 |
| Na Bicarbonate | x | 5 | x | 5 | 10 |
| Dibasic | x | | 5 | X | x |
| Na Phosphate | | | | | |
| Methocel K4M | x | 10 | 5 | X | x |
| Premium | | | | | |
| (Hydroxypropyl- | | | | | |
| methyl Cellulose) | | | | | |
| Methocel K100LV | x | x | 10 | X | x |
| Premium | | | | | |
| (Hydroxypropyl- | | | | | |
| methyl Cellulose) | | | | | |
| Na | x | x | x | 6 | 15 |
| Carboxymethyl- | | | | | |
| cellulose | | | | | |
| (7H3 Aqualon) | | | | | |
| Hydroxyethyl | x | x | x | 3 | x |
| Cellulose (Klucel | | | | | |
| 250M Aqualon) | | | | | |
| Xanthan Gum | 2 | x | x | X | x |
| Microcrystaline | 5 | 10 | 5 | X | x |
| Cellulose | | | | | |
| Polyvinyl | 3 | x | 3 | X | x |
| Pyrrolidone | | | | | |
| Croslinked Na | 2 | x | x | X | x |
| Carboxymethyl | | | | | |
| Cellulose | | | | | |
| Croslinked | x | 2 | 2 | X | x |
| Polyvinyl | | | | | |
| Pyrrolidone¹ | | | | | |
| Sorbitol | 30 | 16.8116 | 19.4448 | 33 | 23 |
| Mannitol | 33.257 | 0 | 0 | 28.49 | 22.49 |
| Cetyl pyrridinium | x | 0.5 | x | X | x |
| Chloride | | | | | |
| Chlorhexidine | x | x | 0.5 | X | x |
| Gluconate | | | | | |
| Zinc Stearate | 1 | 1 | 1 | 1 | 1 |
| Total | 100 | 100 | 100 | 100 | 100 |

¹Plasdone XL from ISP.

2. Mouthrinse Compositions are Described Below:

The following oral care mouthrinse compositions are made by conventional processes by mixing the following:

| Component | EXP 1 | EXP 2 | EXP 3 | EXP 4 | EXP 5 | EXP 6 | EXP 7 | EXP 8 | EXP 9 |
|---|---|---|---|---|---|---|---|---|---|
| Glycerin | 23.000 | 23.000 | 23.000 | 13.000 | 5.000 | 5.000 | 13.000 | 17.000 | 17.000 |
| Cetylpyridinium | 0.040 | 0.065 | 0.070 | 0.050 | 0.045 | 0.075 | 0.065 | 0.075 | x |
| Chloride (CPC) | | | | | | | | | |
| Domiphen | x | x | x | x | 0.005 | x | x | 0.005 | x |
| Bromide (DB) | | | | | | | | | |
| Zinc Lactate | x | x | x | 0.250 | x | x | x | x | x |
| Spearmint - | 0.042 | 0.070 | 0.051 | 0.084 | x | 0.065 | 0.075 | 0.075 | 0.075 |
| natural | | | | | | | | | |
| Spearmint - | x | x | x | x | 0.050 | x | x | x | x |
| synthetic | | | | | | | | | |
| 1-carvone | 0.008 | 0.020 | 0.029 | 0.021 | X | 0.015 | 0.030 | 0.025 | 0.025 |
| Cassia- synthetic | 0.005 | x | 0.010 | 0.010 | 0.005 | 0.010 | 0.010 | 0.010 | 0.010 |
| Cassia - natural | x | 0.010 | x | x | x | x | x | x | x |
| Orange 10x | 0.015 | 0.020 | 0.010 | 0.025 | 0.010 | 0.010 | 0.025 | 0.020 | 0.020 |
| Lemon 10x | 0.010 | 0.020 | 0.005 | 0.010 | 0.010 | 0.005 | 0.035 | 0.015 | 0.015 |
| Menthol | x | 0.020 | 0.015 | 0.010 | 0.005 | 0.015 | 0.015 | 0.015 | 0.015 |
| Saccharin | 0.025 | 0.025 | 0.018 | 0.030 | 0.025 | 0.030 | 0.010 | 0.030 | 0.030 |
| Poloxamer 407 | x | 0.050 | 0.050 | 0.025 | x | 0.050 | 0.050 | 0.025 | 0.025 |
| Monosodium | 0.085 | 0.053 | x | x | x | 0.085 | 0.053 | 0.053 | 0.053 |
| Phosphate | | | | | | | | | |
| Dibasic Sodium | 0.070 | 0.020 | x | x | x | 0.070 | 0.020 | 0.020 | 0.020 |
| Phosphate | | | | | | | | | |
| Color | 0.020 | 0.020 | 0.010 | 0.020 | 0.020 | 0.020 | 0.020 | 0.020 | 0.020 |
| Ethanol | x | x | x | 1.200 | 5.000 | x | x | 12.000 | 12.000 |
| Water | QS | QS | QS | QS | QS | QS | QS | QS | QS |

| | EXP 1 | EXP 2 | EXP 3 | EXP 4 | EXP 5 | EXP 6 | EXP 7 |
|---|---|---|---|---|---|---|---|
| | (% By | (% By | (% By | (% By | (% By | (% By | (% By |
| | Wt. | Wt. | Wt. | Wt. | Wt. | Wt. | Wt. |
| Ingredient | Wet) | Wet) | Wet) | Wet) | Wet) | Wet) | Wet) |
| Water | 70.76% | 71.15% | 60.21% | 76.30% | 70.35% | 70.60% | 72.35% |
| HPMC | 3.00% | 6.00% | 3.00% | 5.00% | 5.00% | 4.00% | 8.00% |
| Methocel | | | | | | | |
| K3¹ | | | | | | | |
| HPMC | x | x | 3.00% | 1.00% | x | 5.00% | x |
| Methocel | | | | | | | |
| E50¹ | | | | | | | |
| HPMC | 2.00% | 2.00% | 2.00% | x | 3.00% | x | 1.00% |
| Methocel | | | | | | | |
| K100¹ | | | | | | | |
| HPMC | 0.50% | 0.50% | x | 0.90% | x | x | 0.90% |
| Methocel | | | | | | | |
| K4M¹ | | | | | | | |
| HPMC | x | x | x | x | 1.00% | x | x |
| Methocel | | | | | | | |
| E4M¹ | | | | | | | |
| Indigestible | 6.50% | 6.30% | 9.00% | 5.50% | 7.00% | 10.50% | 1.00% |
| Dextrin | | | | | | | |
| Acesulfame | 0.50% | 0.80% | 0.90% | 1.80% | 0.90% | x | 0.90% |
| Potassium | | | | | | | |
| Sucralose | x | 0.50% | 0.45% | x | 0.80% | x | 0.90% |
| Dextrin | 1.00% | 3.00% | 5.00% | 1.00% | 5.00% | 0.90% | 1.00% |
| Aspartame | 0.90% | x | 0.50% | x | x | 1.80% | x |
| Citric Acid | 0.50% | 1.00% | 1.10% | 1.00% | 1.00% | 1.00% | 1.00% |
| Spearmint - | 3.10% | x | 2.75% | 1.40% | 1.40% | 1.00% | 4.00% |
| natural | | | | | | | |
| Spearmint - | x | 2.00% | x | x | x | x | x |
| synthetic | | | | | | | |
| 1-carvone | 1.15% | 0.75% | 1.75% | 0.90% | 0.50% | 0.90% | 1.00% |
| Cassia- | 0.50% | 0.35% | 0.50% | x | 0.50% | 0.35% | 0.50% |
| synthetic | | | | | | | |
| Cassia - | x | x | x | 0.50% | x | x | x |
| natural | | | | | | | |
| Orange 10x | 0.75% | 0.80% | 1.25% | 0.70% | 0.35% | 0.15% | 1.00% |
| Lemon 10x | 0.50% | 0.35% | 0.90% | 0.10% | 0.15% | 0.35% | 0.50% |
| Menthol | 1.00% | 0.75% | 0.85% | 0.40% | 0.10% | 0.25% | 0.50% |
| Canola Oil | 2.00% | 1.00% | 2.00% | 0.00% | 0.00% | 0.00% | 0.00% |
| Gum | 2.00% | 1.00% | 2.00% | 2.00% | 1.45% | 1.70% | 2.00% |
| Arabic | | | | | | | |
| Color | 1.00% | 0.75% | 0.50% | 0.50% | 0.50% | 0.50% | 1.25% |
| Sorbitol | 2.34% | 1.00% | 2.34% | 1.00% | 1.00% | 1.00% | 2.20% |
| Total | 100.00% | 100.00% | 100.00% | 100.00% | 100.00% | 100.00% | 100.00% |

To produce the film formulations of examples 1-3, add the film forming agents (Methocel variants) to a mixture containing canola oil, flavoring agent, and sorbitol. Then agitate this mixture until the particles of Methocel powder are homogenously dispersed. Water, at a temperature of approximately 75° C. is then added and agitation is continued for at least 30 minutes. Then add the remaining ingredients, such as color, sweeteners, and the indigestible dextrin, to the solution and mix under agitation for at least 10 minutes. Pour the casting solution onto a glass plate and draw down to form a thin monlayer film. Then dry the film for ten minutes at 70° C. Next, remove the film from the glass plate and cut into the desired dimensions.

To produce the film formulations of examples 4 and 5, heat the water to greater than 180° F. Then add the Methocel variants to the hot water and mix at 180° F. for at least 10 minutes. Follow by adding the remaining ingredients to the hot mixture, such as color, sweeteners, and indigestible dextrin. The mixture is then mixed for at least 5 minutes. Cool the casting solution to 25° C. and pour onto a glass plate and draw down to form a thin monolayer film. Next dry the film for fifteen minutes at 70° C. Next, remove the film from the glass plate and cut into the desired dimensions.

For Examples 6 and 7 thoroughly mix the Methocel variants with dextrin and gum Arabic. Then add this dry mixture to water under high agitation. Continue the agitation for at least 30 minutes. The remaining ingredients, such as color, sweeteners, and the indigestible dextrin, are then added to the solution and mixed under agitation for at least 10 minutes. Next pour the casting solution onto a glass plate and draw down to form a thin monolayer film. Then dry the film for fifteen minutes at 70° C. Next, remove the film from the glass plate and cut into the desired dimensions.

The following method can be used to prepare the films of Examples 8 to 16.
A. The film forming ingredients (e.g. xanthan gum, locust bean gum, carrageenan and pullulan) other than Polysorbate 80 and Atmors 300 are mixed and hydrated in hot purified water to form a gel and stored in a refrigerator overnight at a temperature of approximately 4° C. to form preparation A.
B. The coloring agent(s), copper gluconate and sweetener are added to and dissolved in purified water to form preparation B.
C. Preparation B is added to preparation A and mixed well to form preparation C.
D. The flavoring agents, essential oils, and cooling agents are mixed to form preparation D.
E. The polysorbate 80 and Atmos 300 are added to preparation D and mixed well to from preparation E.
F. Preparation E is added to preparation C and mixed well to form preparation F.

Preparation F is poured on a mold and cast to form a film of a desired thickness at room temperature. The film is dried under warm air and cut to a desired dimension, packaged and stored.

The amounts in these examples are presented as the actual weight (grams) or 20 w/w%. These formulas create the solution/gel that is cast and dried into a film. The actual amount of each ingredient in the finished, dried film depends upon the amount of relative moisture removed during drying.

| | | | EXP | EXP | EXP | EXP | EXP | EXP | EXP |
|---|---|---|---|---|---|---|---|---|---|
| INGREDIENT | EXP 8 | EXP 9 | 10 | 11 | 12 | 13 | 14 | 15 | 16 |
| Xanthan Gum, | 0.03 | 0.03 | 0.06 | 0.03 | 0.03 | 0.03 | 0.06 | 0.06 | 0.06 |
| Locust Bean | 0.07 | 0.07 | 0.07 | 0.07 | 0.07 | 0.07 | 0.07 | 0.07 | 0.07 |
| Carrageenan | 0.3 | 16.0 | 16.0 | 16.0 | 16.0 | 16.0 | 16.0 | 16.0 | 16.0 |
| Pullulan | 16.0 | 16.0 | 16.0 | 16.0 | 16.0 | 16.0 | 16.0 | 16.0 | 16.0 |
| Sodium | 0.1 | 0.1 | 0.1 | 0.07 | 0.07 | 0.07 | 0.07 | 0.07 | 0.07 |
| Benzoate | 0.5 | 0.5 | 0.5 | 0.5 | 0.5 | 0.5 | 0.5 | 0.5 | 0.5 |
| Acesulfame | | | | | | | | | |
| Potassium | 1.4 | 1.4 | 1.4 | 1.4 | 1.4 | 1.4 | 1.4 | 1.4 | 1.4 |
| Aspartame | | | | | | | | | |
| Water | qs 100 | qs | qs 100 | qs 100 | qs | qs 100 | qs | qs 100 | qs 100 |
| | | 100 | | | 100 | | 100 | | |
| Cooling Agent | 0.1 | 0.1 | 0.1 | 0.1 | 0.1 | 0.1 | 0.1 | 0.1 | 0.1 |
| Menthol | 2.0 | 2.0 | x | 2.0 | 2.0 | 2.0 | x | 2.0 | 2.0 |
| Polysorbate 80 | 0.35 | 0.35 | 0.35 | 0.35 | 0.35 | 0.35 | 0.35 | 0.35 | 0.35 |
| Atmos 300 | 0.35 | 0.35 | 0.35 | 0.35 | 0.35 | 0.35 | 0.35 | 0.35 | 0.35 |
| Propylene | 1.0 | 1.0 | 1.0 | 1.0 | 1.0 | 1.0 | 3.0 | 3.0 | 3.0 |
| Glycol | | | | | | | | | |
| PEG 1450 | x | 3.10 | x | x | x | x | x | x | x |
| Olive Oil | x | x | x | 1-2 | 2.0 | 2.0 | 0.5-2.0 | x | 0.5 |
| Polyox N-10 | x | x | x | x | x | x | x | x | 1.0 |
| Titanium | x | 0.25 | 0.25 | 0.25 | 0.25 | x | 0.25 | x | 0.25 |
| Dioxide | | | | | | | | | |
| Spearmint | 0.020 | 0.028 | 0.046 | 0.068 | 0.062 | 0.080 | 0.020 | 0.028 | 0.046 |
| (natural) | | | | | | | | | |
| 1-carvone | 0.007 | 0.022 | 0.024 | 0.005 | 0.081 | x | 0.007 | 0.022 | 0.024 |
| Cassia- | 0.013 | 0.015 | 0.009 | 0.007 | 0.021 | 0.010 | 0.013 | 0.015 | 0.009 |
| (synthetic) | | | | | | | | | |
| Orange 10x | 0.005 | 0.010 | 0.011 | 0.015 | 0.036 | 0.020 | 0.005 | 0.010 | 0.011 |
| Lemon 10x | 0.002 | 0.015 | 0.005 | 0.005 | 0.020 | 0.020 | 0.002 | 0.015 | 0.005 |
| Menthol | 0.003 | 0.010 | 0.005 | x | 0.030 | 0.020 | 0.003 | 0.010 | 0.005 |

4. Whitening Composition:

| Component | EXP 1 | EXP 2 | EXP 3 | EXP 4 | EXP 5 | EXP 6 |
|---|---|---|---|---|---|---|
| Ethyl Acetate | 17.95 | 14.75 | 22.30 | 20.77 | 18.71 | 17.85 |
| 2-Butanone | 18.00 | 13.00 | 13.10 | 20.88 | 10.00 | 18.00 |
| Isododecane | x | 10.00 | x | x | 11.54 | x |
| Limonene | x | 4.35 | x | x | 5.00 | x |
| MQ Resin | 28.00 | 32.50 | 26.50 | 27.33 | 36.00 | 31.50 |
| SE 30 Silicone | 7.00 | x | 8.80 | 13.67 | x | x |
| Gum | | | | | | |
| Silicone | x | x | x | x | x | 3.50 |
| Visc-100M | | | | | | |
| Silicone Fluid | x | 6.50 | x | x | 9.00 | x |
| 10cStk | | | | | | |
| Bentone Gel | 10.00 | x | 6.40 | 9.95 | x | 10.00 |
| ISD | | | | | | |
| Claytone HY | x | 2.45 | x | x | 3.00 | x |
| Cab-o-Sil | x | x | 1.50 | x | x | x |
| Sodium | 19.00 | x | 19.00 | 7.00 | x | 19.00 |
| Percarbonate | | | | | | |
| Carbamide | x | 15.00 | x | x | 5.00 | x |
| Peroxide | | | | | | |
| Bismuth | x | 1.15 | x | x | x | x |
| Oxychloride | | | | | | |
| Titanium | x | x | 1.00 | x | 1.50 | x |
| Dioxide | | | | | | |
| Spearmint | 0.020 | 0.028 | 0.046 | 0.068 | 0.062 | 0.080 |
| (natural) | | | | | | |
| 1-carvone | 0.007 | 0.022 | 0.024 | 0.005 | 0.081 | x |
| Cassia- | 0.013 | 0.015 | 0.009 | 0.007 | 0.021 | 0.010 |
| (synthetic) | | | | | | |
| Orange 10x | 0.005 | 0.010 | 0.011 | 0.015 | 0.036 | 0.020 |
| Lemon 10x | 0.002 | 0.015 | 0.005 | 0.005 | 0.020 | 0.020 |
| Menthol | 0.003 | 0.010 | 0.005 | x | 0.030 | 0.020 |
| Sodium | x | x | 1.00 | x | x | x |
| Fluoride | | | | | | |
| Sodium | x | 0.20 | 0.30 | 0.30 | x | x |
| Saccharin | | | | | | |
| | 100.00 | 100.00 | 100.00 | 100.00 | 100.00 | 100.00 |

The whitening compositions are prepared as follows. Add the solvents into. a container suitable to minimize solvent loss. Add the rheology modifiers and mix until well dispersed. Add the silicone resin and mix until completely dissolved. Add the silicone gum and/or silicone fluids and mix until completely dissolved. At this time add any salts such as sodium percarbonate and/or other oral care actives, aesthetic ingredients such as opacifiers, sweeteners, dyes, and flavors. Continue mixing until homogeneous;. additional high shear mixing may be used, to promote the mixing. Pack into airtight containers.

Alternatively premixes of the silicone resin and/or the silicone gum !nay be prepared prior to incorporation into the final blending step to facilitate silicone dissolution and ease of manufacturing. Depending on the formula composition, the order of ingredient addition may also vary such as the addition of the rheology modifier(s) may be moved to a later step allowing lower viscosity to be maintained until the later stages of the blending step.

The embodiments disclosed and represented by the examples of the previous Tables have many advantages. For example, they can provide better durability and subsequent delivery of oral care substances, particularly to the surfaces of the teeth. They can also provide a convenient, discrete, and easy to use product form which can deliver benefits that are significantly different from those that can be achieved by conventional product forms. In addition, oral care actives that would exhibit instability in an aqueous-based film system can be incorporated into the compositions herein without compromising stability.

Method of Use

The tooth whitening composition can be applied with a brush, a pen applicator, a doe's foot applicator, or the like, or even with the fingers.

A film containing the tooth whitening substance quickly forms on the surface to which the composition has been applied. Prolonged delivery of the oral care substance is made possible as the oral care substance is released from the film over time. Then, any residual oral care substance may be easily removed by wiping, brushing or rinsing the oral surface after a desired period of time has elapsed, or in the normal course of tooth brushing or other oral care activities. Without being bound by theory, it is believed that the film will last from about 2 hours to 8 hours regardless of the reactivity of the oral care substance. Preferably, the compositions are almost unnoticeable when applied to the oral cavity.

5. Gums

| Material Name | | | EXP 3 | | EXP 4 | |
|---|---|---|---|---|---|---|
| (INCI) | EXP 1 | EXP 2 | Core | Coating | Core | Coating |
| Sorbitol | 25.850 | 18.350 | 33.350 | x | 33.350 | x |
| Xylitol | 16.700 | 16.700 | 16.700 | x | 16.700 | x |
| Gum base (e.g. | 28.000 | 28.000 | 28.000 | x | 28.000 | x |
| Prestige-PL, | | | | | | |
| Cafosa) | | | | | | |
| Sodium | 7.500 | 15.000 | 0.000 | 37.500 | 0.000 | x |
| polyphosphate, | | | | | | |
| n = 21; Glass | | | | | | |
| H (FMC) | | | | | | |
| Glass H | x | x | x | x | x | 37.500 |
| (microporous | | | | | | |
| sized) | | | | | | |
| Hydrogenated | 8.000 | 8.000 | 8.000 | x | 8.000 | x |
| starch | | | | | | |
| hydrolysate | | | | | | |
| (85% solids) | | | | | | |
| Glycerin | 7.000 | 7.000 | 7.000 | x | 7.000 | x |
| Mannitol | 5.000 | 5.000 | 5.000 | x | 5.000 | |
| Maltitol | x | x | x | 62.250 | x | 62.250 |
| Ethyl cellulose | x | x | x | x | x | x |
| (Ethocell, Dow | | | | | | |
| Corning) | | | | | | |
| Spearmint - | 0.850 | 0.450 | 0.720 | 0.09 | 0.950 | 0.170 |
| natural | | | | | | |
| 1-carvone | 0.350 | 0.750 | 0.480 | 0.08 | x | x |
| Cassia- | 0.100 | 0.050 | 0.100 | 0.01 | 0.150 | 0.010 |
| synthetic | | | | | | |
| Orange 10x | 0.100 | 0.150 | 0.100 | 0.03 | 0.200 | 0.030 |
| Lemon 10x | 0.100 | 0.100 | 0.100 | 0.02 | 0.200 | 0.020 |
| Menthol | 0.100 | 0.100 | 0.100 | 0.02 | 0.100 | 0.020 |
| Aspartame | 0.200 | 0.200 | 0.200 | x | 0.200 | x |
| Spray dried | 0.150 | 0.150 | 0.150 | x | 0.150 | x |
| flavor | | | | | | |
| TOTAL | 100.0 | 100.0 | 100.0 | 100.00 | 100.0 | 100.0 |
| % OF TOTAL | 100.0 | 100.0 | 80.00 | 20.000 | 80.00 | 20.00 |
| COMPOSI- | | | | | | |
| TION | | | | | | |

### Examples 1, 2

Heat gum base to ˜45° C. to soften. Maintain mixer vessel cavity at ˜45° C. during entire mixing process. Add gum base to mixing cavity of double sigma blade mixer and mix for 5 minutes. Add mannitol and spray-dried menthol. Mix for 2 minutes. Add glycerin and mix for 2 minutes. Add 50% of xylitol and mix for 2 minutes. Add hydrogenated starch hydrolysate and mix for 5 minutes. Add 50% sorbitol and mix for 3 minutes. Add second 50% of xylitol, Glass-H (if present in the composition) and aspartame and mix for 3 minutes. Add flavor and mix for 3 minutes.

### Examples 3, 4

Heat gum base to −45° C. to soften. Maintain mixer vessel cavity at −45° C. during entire mixing process. Add gum base to mixing cavity of double sigma blade mixer and mix for 5 minutes. Add mannitol and spray-dried menthol. Mix for 2 minutes. Add glycerin and mix for 2 minutes. Add 50% of xylitol and mix for 2 minutes. Add hydrogenated starch hydrolysate and mix for 5 minutes. Add 50% sorbitol and mix for 3 minutes. Add second 50% of xylitol aspartame-and mix for 3 minutes. Add flavor and mix for 3 minutes. Store for 1 week at 15-20° C., 30-60% RH (conditioning). Tumble chewing gums in coater-drier with maltitol solution and Glass-H. Maintain tumbling until surface is dry.

6. Lozenges

| INGREDIENTS | EXP 1 | EXP 2 | EXP 3 | EXP 4 |
|---|---|---|---|---|
| Isomalt | 97.055 | 96.805 | 96.555 | 96.055 |
| Water | 2.400 | 2.400 | 2.400 | 2.400 |
| Acelsulfame K | 0.045 | 0.045 | 0.045 | 0.045 |
| Spearmint - | 0.2085 | x | 0.417 | 0.6255 |
| Natural | | | | |
| Spearmint- | x | 0.31275 | x | x |
| Synthetic | | | | |
| L-Carvone | 0.075 | 0.1125 | 0.15 | 0.225 |
| Cassia - Natural | 0.0335 | x | 0.067 | 0.1005 |
| Cassia - Synthetic | x | 0.05025 | x | x |
| Orange Oil | 0.058 | 0.087 | 0.116 | 0.174 |
| Lemon Oil | 0.025 | 0.0375 | 0.05 | 0.075 |
| Menthol | 0.100 | 0.15 | 0.20 | 0.30 |

### Example 1

Isomalt is dissolved in water and heated under stirring to 110-112° C. and subsequently cooked to 141-145° C. to boil off water. Batch is drawn down under vacuum and essential oils added at approximately 90° C. in low humidity environment. Batch is folded on a hot table and subsequently cooled on cold table (20° C.) prior to transfer to the batch forming and die cutting apparatus. Final product has a Glass like transiupcedtapp earacne.

### Examples 2-3

Low boiled candy: Isomalt is slowly dissolved in water to 80° C. and subsequently cooked to 118-130° C. at which point the heating is removed. Butter or other fat is added 2-3° C. lower than this final cook temperature. Gelatine, essential oils, acid and actives are added afier cooking has ended following which the final products are formed.

### Example 4

Isomalt is dissolved in water and heated under stirring to 110-112° C. Glucose syrup is then added and the mix heated to 141-142° C. to boil off water. The batch is drawn down under vacuum and actives/essential oils added at approximately 90° C. in low humidity environment. The batch is folded on a hot table and subsequently cooled on a cold table (20° C.)prior trinsfer to the batch forming and die cutting apparatus. Final product has a glass like translucent appearance.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An essential oil composition comprising:
(a) two or more essential oils selected from the following Group A essential oils: spearmint, peppermint, a botanical of the Labiatae family, carvone, cinnamon, cassia, clove, anise, ginger, pepper, nutmeg, allspice, and coriander; and
(b) one or more essential oils selected from the following Group B essential oils: lemon, lime, orange, grapefruit, tangerine, mandarin, Ugli fruit, bergarhot, and tomato; wherein the ratio of Group A essential oils to Group B essential oils is from about 2:1 to about 10:1.

2. A consumer product comprising the essential oil composition of claim 1.

3. The consumer product of claim 2, wherein said ratio is from about 2:1 to about.6:1.

4. The consumer product of claim 2, wherein said ratio is from about 3:1 to about 5:1.

5. The consumer product of claim 2, wherein said Group A essential oils are spearmint and cinnamon.

6. The consumer product of claim 2, wherein said Group B essential oil is orange.

7. An oral care composition comprising:
(a) two or more essential oils selected from the following Group A essential oils: spearmint, peppermint, a botanical of the Labiatae family, carvone, cinnamon, cassia, clove, anise, ginger, pepper, nutmeg, allspice, and coriander;
(b) one or more essential oils selected from the following Group B essential oils: lemon, lime, orange, grapefruit, tangerine, mandarin, Ugli fruit, bergamot, and tomato; and
(c) an oral care carrier selected from the group consisting of dentifrice, chewable dentifrice tablet, gels, rinses, edible films, candy, confectionary, gums, and lozenges.
wherein the ratio of Group A essential oils to Group B essential oils is from about 2:1 to about 10:1

8. The oral care composition of claim 7, wherein said oral care carrier is a dentifrice.

9. The oral care composition of claim 7, wherein said oral care carrier is a chewable dentifrice tablet.

10. The oral care composition of claim 7, wherein said oral care carrier is a rinse.

11. The oral care composition of claim 7, wherein said oral care carrier is a gel.

12. The oral care composition of claim 7, wherein said oral care carrier is an edible film.

13. The oral care composition of claim 7, wherein said oral care carrier is a candy.

14. The oral care composition of claim 7, wherein said oral care carrier is a confectionary.

15. The oral care composition of claim 7, wherein said oral care carrier is a gum.

16. The oral care composition of claim 7, wherein the Group A essential oils are spearmint and cassia.

17. The oral care composition of claim 7, comprising two Group B essential oils.

18. The oral care composition of claim 13, wherein said two Group B essential oils are lemon and orange.

19. An oral care composition comprising:
(a) an essential oil composition, wherein said essential oil composition comprises at least one mint essential oil, at least one spice essential oil, and at least one citrus essential oil; wherein said essential oils are added at a ratio of from about 6 mint: 1 spice: 1 citrus to about 2 mint: 1 spice: 1 citrus; and
(b) an oral care carrier selected from the group consisting of dentifrice, gels, rinses, edible films, candy, confectionary, gums, and lozenges; and
(c) an oral care active.

20. The oral care composition of claim 19, wherein said at least one mint essential oil comprises spearmint, and wherein said at least one spice essential oil comprises cassia, and wherein said oral care composition comprises two citrus essential oils selected from the group consisting of lemon, orange, grapefruit, tomato and combinations thereof.
